(19) United States
(12) Patent Application Publication
(10) Pub. No.: US 2006/0270899 A1
(43) Pub. Date: Nov. 30, 2006

(54) Magnetic pill with camera and electrical properties

(76) Inventor: Omar Amirana, Boston, MA (US)

Correspondence Address: VIERRA MAGEN MARCUS & DENIRO LLP, 575 MARKET STREET SUITE 2500, SAN FRANCISCO, CA, 94105, US

(21) Appl. No.: 11/435,320

(22) Filed: May 15, 2006

Related U.S. Application Data
(60) Provisional application No. 60/681,217, filed on May 13, 2005.

(51) Int. Cl. A61B 1/00

(52) U.S. Cl. 600/102

(57) ABSTRACT

A magnetic device (“Pill”) that can be remotely maneuvered and positioned within the GI Tract using an external magnetic field.

### CLAIM OF PRIORITY

This application claims the benefit of U.S. Provisional Application No. 60/681,217 filed May 13, 2005, which application is incorporated by reference in its entirety herein.

### BACKGROUND OF THE INVENTION

Endoscopy has been performed millions of times annually for many diagnostic and therapeutic purposes. It has many drawbacks and limitations including the difficulty or inserting a long endoscope into the gastrointestinal tract. New advances in technology allow for miniaturization of electronics and allow for remote manipulation of devices inside the body. The ability to visualize the gastrointestinal tract remotely with a small ingestible device that is capable of being remotely maneuvered and positioned is unique and valuable for both diagnostic and therapeutic purposes.

In the gastrointestinal arena, stimulators are being used to help control bowel function with applications in obesity, ileus, gastroparesis and dysmotility syndromes. To date, all of the devices addressing these clinical problems require a pacemaker like device attached to a lead. In addition, the devices need to be either surgically implanted or implanted via a minimally invasive procedure. All of these devices rely on electrical stimulation in order to affect the bowel. To the best of my knowledge, no device exists to date which is ingestible, capable of being remotely activated and programmed which also produces a therapeutic effect from within the lumen of the bowel.

### BRIEF DESCRIPTION OF THE DRAWING

The present invention will now be described with reference to the drawings, in which:

FIG. 1 illustrates a maneuverable ingestible device according to an embodiment of the present invention.

FIG. 2 illustrates an external magnetic field system according to an embodiment of the present invention.

### SUMMARY OF THE INVENTION

Magnetic device (“Pill”) that can be remotely maneuvered and positioned within the GI Tract using an external magnetic field. Includes:

Pill that is maneuvered and positioned magnetically and is capable or capturing images and video data.

Pill that is maneuvered and positioned magnetically and is capable or capturing images and video data that can be wirelessly transmitted from the body and processed and evaluated remotely.

Pill that is maneuvered and positioned magnetically and can electrically activate local tissue.

Pill that is maneuvered and positioned magnetically and can electrically damage tissue locally.

Pill that is maneuvered and positioned magnetically, can store and release medication, and can produce local electroporation at a desired location to enhance medication uptake into the tissue.

Pill that is maneuvered and positioned magnetically and can be held in place for therapeutic effects by a wearable magnetic field (such as a wearable vest or girdle that produces adjustable magnetic field).

Pill(s) that is (are) maneuvered and positioned magnetically and can produce electrical signals that are recorded on the skin or remotely to calculate the Pill's location in time and space for 3D reconstruction of the GI Tract, also allowing for electrical activity of the GI tract as real images to be superimposed on the GI tract reconstruction.

Pill that is maneuvered and positioned magnetically and contains ionizing radiation element(s) to injure or destroy tissue a given site.

Pill that is maneuvered and positioned magnetically and contains ionizing radiation element(s) that have electrode(s) capable producing electrical signal(s) and stimulus(i).

Pill that is maneuvered and positioned magnetically and contains ionizing radiation elements that store and selectively release medication.

### DETAILED DESCRIPTION

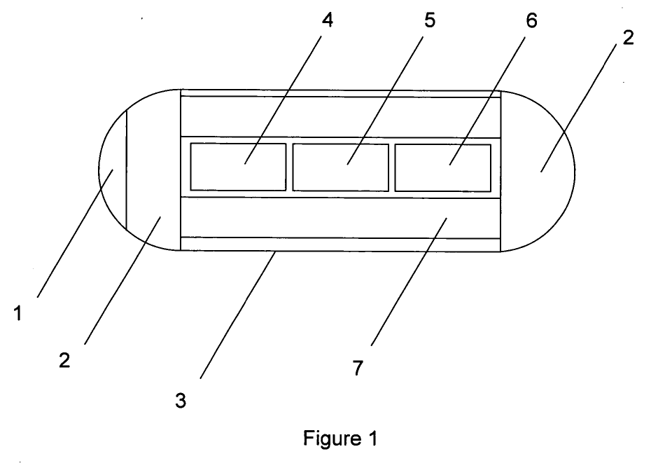

The invention shown in FIG. 1 is a programmable, controllable, maneuverable ingestible device (one capable of safely being swallowed, whether absorbed or not or excreted or not, hereafter referred to as a “Pill”) which contains one or multiple rod-shaped magnetic element(s) (7) capable of being remotely manipulated when located inside the gastrointestinal tract by an external electromagnetic or magnetic field. The magnetic element in the device is a critical part of the invention as it enables the physician to remotely control the transmission and the position of the device as it traverses the patient's gastrointestinal system. Without a magnetic element the physician loses the resulting ability to maneuver and position the Pill, making various diagnostic assessments and targeting various therapies highly inefficient, difficult, cumbersome, improbable or impossible.

In addition to the magnetic element used to maneuver and position the Pill, it also contains some or all of the following characteristics: a non conducting physiologically inert outer casing (3), battery (5), a camera (4) capable of capturing image (static and/or video) data through an aperture (1), circuitry capable of storing, transmitting and receiving data such as image, electrical and physiologic data (6), electrodes (2) and circuitry (6) capable of sensing and producing electrical signals, whether direct current or alternating current, whether intermittent or continuous, whether or not capable of stimulating the gastrointestinal tract or simply for diagnostic purposes such as localization, whether or not to produce local electroporation to enhance drug uptake in a given region of tissue, circuitry enabling remote activation and deactivation as well as programmability, radioactive elements for various uses such as cancer therapy, drug delivery capability to specifically release medication or compounds into the gastrointestinal tract at a given location, Pill to Pill as well as Pill to System wireless communication for timed or gated diagnostic or therapeutic actions using multiple Pills such as, but not limited to, physiologic data capture including images, or for drug release or for electrical activity including gastrointestinal stimulation.

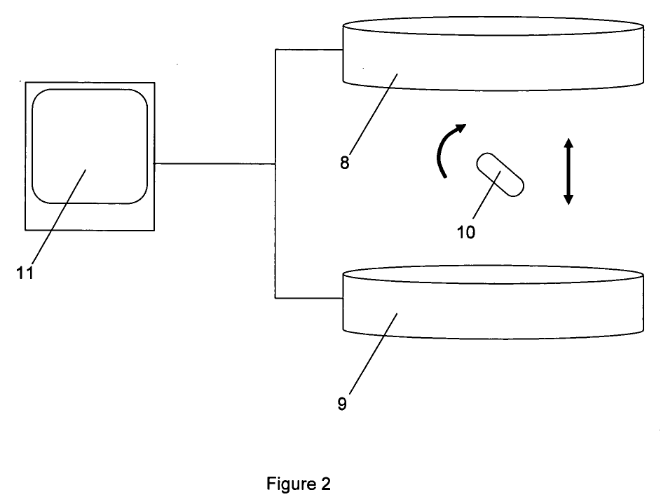

As shown in FIG. 2, the system also includes an external magnetic field system comprising one or more elements that create an external magnetic field of varying strength which imparts a force on the Pill (10) in order to move it or keep in one location while in the gastrointestinal tract for diagnostic and therapeutic purposes. The external magnetic field is controlled by a workstation (11).

Essentially, this invention is an ingestible, magnetically guided device that would obviate the need to use an endoscope which could also include various additional diagnostic and therapeutic features. One version of the device is essentially an ingestible, remotely maneuverable, leadless GI stimulator where the device is the stimulator and the lead all contained in one.

Variety of Features and unique characteristics:
1. Pill with embedded electrical stimulation.
2. Pill with embedded electrical stimulation that is activated by a non-invasive and non-contact means such as an RF transmitter and receiver apparatus.
3. Pill with embedded electrical stimulation that is activated and programmable by a non-invasive and non-contact means such as an RF transmitter and receiver apparatus.
4. Pill with embedded electrical stimulation capability for the purposes of promoting or producing gastrointestinal movement, stimulation, or motility promotion.
5. Pill with embedded electrical stimulation capability for the purposes of treating and managing ileus, constipation and/or dysmotility disorders including obstructive bowel disorders.
6. Pill with embedded electrical stimulation capability and drug delivery capability.
7. Pill with embedded electrical stimulation capability and drug delivery capability for the purposes of gastrointestinal and bowel movement, stimulation, or motility promotion.
8. Pill with embedded mechanical stimulation, vibration and electrical stimulation capability.
9. Pill with embedded mechanical stimulation, vibration, electrical stimulation and drug delivery capability.
10. Pill with embedded mechanical stimulation, vibration, electrical stimulation and drug delivery capability for the purposes of promoting or producing gastrointestinal movement, stimulation, or motility promotion.
11. Pill with embedded electrical stimulation capability and camera which wirelessly transmits pictures to viewing system.
12. Pill with embedded mechanical stimulation, vibration, and/or electrical stimulation and/or drug delivery capability and camera which wirelessly transmits pictures to viewing system.
13. Pill with embedded mechanical stimulation, vibration, and/or electrical stimulation and/or drug delivery capability and camera which wirelessly transmits pictures to viewing system for the purposes of promoting or producing gastrointestinal movement, stimulation, or motility promotion.
14. Pill with embedded mechanical stimulation, vibration, and/or electrical stimulation and/or drug delivery capability capable of electrical or physiological parameter sensing and assessment.
15. Pill with embedded mechanical stimulation, vibration, and/or electrical stimulation and/or drug delivery capability capable of electrical or physiological parameter sensing and assessment where one of the parameters detected is bowel activity and GI motility.
16. Pill with any or all of the following: embedded mechanical stimulation, vibration, electrical stimulation, drug delivery capability, electrical or physiological parameter sensing and assessment where pH is the parameter being measured.
17. Pill with any or all of the following: embedded mechanical stimulation, vibration, electrical stimulation, drug delivery capability, electrical or physiological parameter sensing and assessment along with an embedded identification signature such as RFID tag.

Although the invention has been described in detail herein, it should be understood that the invention is not limited to the embodiments herein disclosed. Various changes, substitutions and modifications may be made thereto by those skilled in the art without departing from the spirit or scope of the invention as described and defined by the appended claims.

I claim:

1. A system including an ingestible device with magnetic properties wherein the ingestible device can be remotely and selectively maneuvered and positioned within the gastrointestinal tract using an external system involving an electromagnetic field including but not limited to embodiments
a. which can be remotely activated and deactivated,
b. which can be remotely programmed
c. which can remotely transmit and receive data including commands

2. A system according to claim 1 wherein the ingestible device is capable of being remotely and selectively maneuvered and positioned in one location within the gastrointestinal tract using an external system using an external electromagnetic field.

3. A system according to claim 1 wherein the ingestible device has the ability to capture and store or capture and wirelessly transmit image data of the gastrointestinal tract.

4. A system according to claim 3 wherein the ingestible device has the ability to capture and store or capture and wirelessly transmit image data of the gastrointestinal tract where the image data is video data.

5. A system according to claim 3 wherein the ingestible device has the ability to capture and store or capture and wirelessly transmit image data of the gastrointestinal tract where the transmitted data is captured and processed by an external data processing system for evaluation system after the procedure.

6. A system according to claim 3 wherein the ingestible device has the ability to capture and store or capture and wirelessly transmit image data of the gastrointestinal tract where the transmitted data is captured and processed by an external data processing system for evaluation system in real time.

7. A system according to claim 1 wherein the ingestible device has the ability to store and selectively release medication or compounds into gastrointestinal tract.

8. A system according to claim 3 in which the ingestible device has the ability to store and selectively release medication or compounds into gastrointestinal tract.

9. A system according to claim 1 wherein the ingestible device has one or a plurality of electrodes capable of sensing and storing or sensing and transmitting an electrical signal or signals.

10. A system according to claim 3 wherein the ingestible device has one or a plurality of electrodes capable of sensing and storing or sensing and transmitting an electrical signal or signals.

11. A system according to claim 1 wherein the ingestible device has one or a plurality of electrodes capable of producing an electrical signal or signals.

12. A system according to claim 3 wherein the ingestible device has one or a plurality of electrodes capable of producing an electrical signal or signals.

13. A system according to claim 1 wherein the ingestible device has one or a plurality of electrodes capable of producing an electrical signal or signals where the electrical signal is a direct current signal either of continuous or intermittent duration such as a pulsed signal.

14. A system according to claim 3 wherein the ingestible device has one or a plurality of electrodes capable of producing an electrical signal or signals where the electrical signal is a direct current signal either of continuous or intermittent duration such as a pulsed signal.

15. A system according to claim 1 wherein the ingestible device has one or a plurality of electrodes capable of producing an electrical signal or signals where the electrical signal is an alternating current signal either of continuous or intermittent duration such as a pulsed signal.

16. A system according to claim 3 wherein the ingestible device has one or a plurality of electrodes capable of producing an electrical signal or signals where the electrical signal is an alternating current signal either of continuous or intermittent duration such as a pulsed signal.

17. A system according to claim 1 wherein the ingestible device has one or a plurality of electrodes capable of both sensing and producing an electrical signal or signals.

18. A system according to claim 3 wherein the ingestible device has one or a plurality of electrodes capable of both sensing and producing an electrical signal or signals.

19. A system according to claim 1 wherein the ingestible device has one or a plurality of electrodes capable of sensing and producing an electrical signal or signals where the electrical signal is a current signal either of continuous or intermittent duration such as a pulsed signal which is capable of being sensed by sensors placed on the skin, including but not limited to:
a. an embodiment where the signal is sensed on the skin at multiple locations simultaneously
b. an embodiment where the signal is sensed on the skin at multiple locations simultaneously to calculate the three dimensional position of the ingestible device at a given point in time and space
c. an embodiment where the signal(s) are sensed on the skin at multiple locations simultaneously to calculate the three dimensional position of the ingestible device at a given point in time and space, and this is performed numerous times in order to reconstruct a three dimensional image of the lumen of the gastrointestinal tract
d. an embodiment where the signal(s) are sensed on the skin at multiple locations simultaneously to calculate the three dimensional position of the ingestible device at a given point in time and space, and this is performed numerous times in order to reconstruct a three dimensional image of the lumen of the gastrointestinal tract and also to simultaneously superimpose the gastrointestinal tract's electrical activity onto the three dimensional reconstruction.

20. A system according to claim 1 wherein the ingestible device has one or a plurality of electrodes capable of sensing and producing an electrical signal or signals where the electrical signal is a current signal either of continuous or intermittent duration such as a pulsed signal which is capable of being sensed by sensors placed on the skin, including but not limited to:
a. an embodiment where the signal is sensed on the skin at multiple locations simultaneously
b. an embodiment where the signal is sensed on the skin at multiple locations simultaneously to calculate the three dimensional position of the ingestible device at a given point in time and space
c. an embodiment where the signal(s) are sensed on the skin at multiple locations simultaneously to calculate the three dimensional position of the ingestible device at a given point in time and space, and this is performed numerous times in order to reconstruct a three dimensional image of the lumen of the gastrointestinal tract
d. an embodiment where the signal(s) are sensed on the skin at multiple locations simultaneously to calculate the three dimensional position of the ingestible device at a given point in time and space, and this is performed numerous times in order to reconstruct a three dimensional image of the lumen of the gastrointestinal tract and also to simultaneously superimpose the gastrointestinal tract's electrical activity onto the three dimensional reconstruction or to superimpose the gastrointestinal tract's visual appearance onto the three dimensional reconstruction.
